(19) United States
(12) Patent Application Publication
(10) Pub. No.: US 2016/0271114 A1
(43) Pub. Date: Sep. 22, 2016

(54) Composition for preventing or treating bacterial infectious disease comprising phospholipase D2 inhibitor

(71) Applicant: RESEARCH & BUSINESS FOUNDATION SUNGKYUNKWAN UNIVERSITY, Suwon-si (KR)

(72) Inventors: Yoe-Sik BAE, Seongnam-si (KR); Sung Kyun LEE, Suwon-si (KR)

(73) Assignee: RESEARCH & BUSINESS FOUNDATION SUNGKYUNKWAN UNIVERSITY, Suwon-si (KR)

(21) Appl. No.: 15/065,300

(22) Filed: Mar. 9, 2016

(30) Foreign Application Priority Data
Mar. 9, 2015 (KR) 10-2015-0032318

(51) Int. Cl. A61K 31/435

(52) U.S. Cl. CPC A61K 31/435

(57) ABSTRACT

Provided are a composition for preventing or treating a bacterial infectious disease, comprising a phospholipase D2 (PLD2) inhibitor as an active ingredient and a method for treating the bacterial infectious disease using the same.

Since the PLD2 inhibitor according to the present invention exhibits characteristics such as blocking of lung inflammation and liver inflammation, bactericidal activity through induction of NET production, effective maintenance of neutrophil mobility through the blocking of intracellular CXCR2 migration, and blocking of the production of inflammatory cytokines in bacterial infectious disease models, particularly, sepsis models, it is expected to be used as a therapeutic agent for sepsis or septic shock.

The present invention was supported by Project No. 1465016273 of the disease-based translational research project funded by the Ministry of Health and Welfare.

### CROSS-REFERENCE TO RELATED APPLICATION

This application claims priority to and the benefit of Korean Patent Application No. 10-2015-0032318, filed on Mar. 9, 2015, the disclosure of which is incorporated herein by reference in its entirety.

### BACKGROUND

1. Field of the Invention

The present invention relates to a composition for preventing or treating a bacterial infectious disease comprising a phospholipase D2 (PLD2) inhibitor as an active ingredient and a treatment method using the same.

2. Discussion of Related Art

Sepsis, which is one of the bacterial infections, is a systemic inflammatory response syndrome (SIRS) generated from the infection of invading microorganisms (Cohen, J., 2002, “The immunopathogenesis of sepsis,” Nature, 420:885-891.) Sepsis may be caused by the infiltration of microorganisms coexisting in an adjacent tissue from the gastrointestinal tract or skin, and the partial infection of the urogenital organs, the gall bladder, the lung, the gastrointestinal tract, etc. may induce blood infection. Furthermore, microorganisms may infiltrate into the blood through intravenous injection, etc. That is, due to the infection of microorganisms, various types of responses of a host, that is, a human, are exhibited, and indicated by inflammatory responses such as high temperature or low temperature, chills, tachycardia, tachypnea, etc. Sepsis is a very deadly disease that develops into severe sepsis, septic shock, or a multiple organ dysfunction syndrome (MODS) causing dysfunction in the lung, kidney, liver, circulatory system, etc. as a complication, unless the cause of the disease is quickly and exactly diagnosed at an early stage. During the past two decades, despite the progressive development of the medical field, the occurrence frequencies of severe sepsis and septic shock are continuously increasing, and more than 750,000 new sepsis patients are identified every year in the United States. Overall mortality associated with severe sepsis in the United States is about 27%, in which the in-hospital mortality is 17%. However, currently, there is no effective medicine capable of prolonging the life of a sepsis patient.

A recent study found that the mortality due to sepsis is closely associated with the inability to regulate inflammatory responses caused by innate immune system disorders in early sepsis. Also, sepsis is accompanied with excessive lymphocyte apoptosis, and therefore, multi-organ failure occurs. A noticeable change in cytokine levels occurs, and proinflammatory cytokines, for example, tumor necrosis factor-α (TNF-α), IL-1β, etc. significantly increase. Therefore, bactericidal activity, the prevention of an imbalance of cytokines, the inhibition of apoptosis of lymphocytes or death should be a target for sepsis treatment.

Meanwhile, PLD2 is an enzyme for hydrolyzing phosphatidylcholine into phosphatidic acid (PA) and choline (Jang, J. H., C. S. Lee, D. Hwang, and S. H. Ryu, 2012, “Understanding of the roles of phospholipase D and phosphatidic acid through their binding partners,” Progress in Lipid Research, 51:71-81.) Currently, it has been reported that PLD2 is associated with various biological processes such as vesicle transport, cell migration through a cytoskeleton, cell proliferation/apoptosis and cell differentiation (Jang, J. H., C. S. Lee, D. Hwang, and S. H. Ryu, 2012, “Understanding of the roles of phospholipase D and phosphatidic acid through their binding partners,” Progress in Lipid Research, 51:71-81). However, the function and role of PLD2 in the pathogenesis of sepsis have not been disclosed.

### SUMMARY OF THE INVENTION

The inventors had identified the function of PLD2 during the pathological progression of sepsis by the result of a study for developing an effective medicine for a bacterial infectious disease, particularly, sepsis or septic shock, and thus completed the present invention.

The present invention is directed to providing a composition for preventing, relieving or treating a bacterial infectious disease, which comprises a PLD2 inhibitor as an active ingredient.

However, technical problems to be solved in the present invention are not limited to the above-described problems, and other problems which are not described herein will be fully understood by those of ordinary skill in the art from the following descriptions.

In one aspect, the present invention provides a pharmaceutical composition for preventing or treating a bacterial infectious disease, which comprises a PLD2 inhibitor as an active ingredient.

According to an exemplary embodiment of the present invention, the disease may be sepsis or septic shock.

According to another exemplary embodiment of the present invention, the PLD2 inhibitor may be selected from the group consisting of 5-fluoro-2-indolyl des-chlorohalopemide, N-[2-[1-(3-Fluorophenyl)-4-oxo-1,3,-8-triazaspiro[4.5]dec-8-yl]ethyl]-2-naphthalenecarboxamide, N-{2-[4-oxo-1-phenyl-1,3,8-triazaspiro(4.5)decan-8-yl]ethyl}quinoline-3-carboxamide, N-(2-{4-[2-oxo-2,3-dihydro-1H-benzo(d)imidazol-1-yl]piperidin-1-yl}ethyl)-2-naphthamide, (1R,2R)—N—([S]-1-{4-[5-bromo-2-oxo-2,3-dihydro-1H-benzo(d)imidazol-1-yl]piperidin-1-yl}propan-2-yl)-2-phenylcyclopropanecarboxamide), and N-[2-[4-(5-chloro-2, 3-dihydro-2-oxo-1H-benzimidazol-1-yl)-1-piperidinyl]-ethyl]-4-fluoro-benzamide.

According to still another exemplary embodiment of the present invention, the composition may have bactericidal activity.

According to yet another exemplary embodiment of the present invention, the composition may enhance peptidylarginine deiminase 4 (PAD4) activity in neutrophils.

According to yet another exemplary embodiment of the present invention, the composition may decrease the production of inflammatory cytokines.

According to yet another exemplary embodiment of the present invention, an inflammatory cytokine may be selected from the group consisting of TNF-α, IL-1β, IFN-γ, IL-17 and IL-23.

According to yet another exemplary embodiment of the present invention, the composition may inhibit apoptosis of lymphocytes.

In another aspect, the present invention provides a health functional food composition for preventing or relieving a bacterial infectious disease, comprising a PLD2 inhibitor as an active ingredient.

In still another aspect, the present invention provides a method for treating a bacterial infectious disease, which includes administering a PLD2 inhibitor into a subject.

In yet another aspect, the present invention provides a use of a PLD2 inhibitor for preventing, relieving or treating a bacterial infectious disease.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows (A) the variations of survival rates over time after WT and PLD2 knockout (KO) mice are subjected to CLP surgery, (B) the variations of survival rates over time after a CLP surgery is performed on WT mice and then the mice are treated with a vehicle or a PLD2 inhibitor, and (C) the variations of survival rates in S. aureus-induced sepsis models;

FIG. 2 shows (A) the results of histological analysis for the lungs extracted from WT and PLD2 knockout (KO) mice subjected to sham or CLP surgery, and (B) the quantification results obtained by measuring the wet to dry (W/D) weight ratios of the lungs extracted from WT and PLD2 knockout (KO) mice subjected to sham or CLP surgery;

FIG. 3 shows (A) the results of histological analysis for livers extracted from WT and PLD2 knockout (KO) mice subjected to CLP surgery, and (B) the AST results for WT and PLD2 knockout (KO) mice subjected to CLP surgery;

FIG. 4 shows the results obtained by the expression of inflammatory cytokines and chemokines in the peritoneal lavage fluids of WT and PLD2 knockout (KO) mice subjected to CLP surgery;

FIG. 5 shows (A) the comparison of the variations of expression of inflammatory cytokines and chemokines when spleen cells isolated from WT and PLD2 knockout (KO) mice are stimulated with LPS, and (B) the comparison of the variations of expression of inflammatory cytokines and chemokines when spleen cells isolated from WT mice are treated with a vehicle or a PLD2 inhibitor and stimulated with LPS;

FIG. 6 shows the apoptosis in the thymus and spleen isolated from WT and PLD2 knockout (KO) mice subjected to sham or CLP surgery;

FIG. 7 shows the apoptosis in the kidneys and livers isolated from WT and PLD2 knockout (KO) mice subjected to sham or CLP surgery;

FIG. 8 shows the apoptosis in the spleens isolated from WT and PLD2 knockout (KO) mice subjected to sham or CLP surgery;

FIG. 9 shows the comparison of bacterial colony counts according to PLD2 deficiency in the peritoneal lavage fluids, peripheral bloods, bronchoalveolar lavage fluids, lungs, livers and spleen tissues, which are isolated from WT and PLD2 knockout (KO) mice subjected to CLP surgery;

FIG. 10 shows (A) the comparison of NET formation after neutrophils isolated from a bone marrow of WT or PLD2 knockout (KO) mice are stimulated with ionomycin, and (B) the comparison of NET formation after neutrophils isolated from the bone marrow of WT mice are treated with a vehicle or a PLD2 inhibitor and stimulated with ionomycin;

FIG. 11 shows the comparison of citrullinated histone 3 levels after neutrophils isolated from the bone marrow of WT or PLD2 knockout (KO) mice are stimulated with ionomycin;

FIG. 12 shows the comparison of citrullinated histone 3 levels in the peritoneal fluids isolated from WT and PLD2 knockout (KO) mice subjected to CLP surgery;

FIG. 13 shows the comparison of citrullinated histone 3 levels in the lungs isolated from WT and PLD2 knockout (KO) mice subjected to CLP surgery;

FIG. 14 shows (A) the comparison of PAD activities measured after neutrophils isolated from WT or PLD2 knockout (KO) mice are stimulated with ionomycin, and (B) the comparison of PAD activities in cells isolated from lungs of WT and PLD2 knockout (KO) mice subjected to CLP surgery;

FIG. 15 shows the comparison of intracellular calcium mobilization after neutrophils isolated from WT or PLD2 knockout (KO) mice are stimulated with ionomycin;

FIG. 16 shows (A) the comparison of the total number of white blood cells in bronchoalveolar lavage fluids (BALFs) isolated from WT or PLD2 knockout (KO) mice subjected to CLP surgery, and (B) the comparison of the numbers of neutrophils and monocytes;

FIG. 17 shows (A) the comparison of expression levels of CXCR2 in neutrophils of bronchoalveolar lavage fluids (BALFs) isolated from WT or PLD2 knockout (KO) mice subjected to CLP surgery, (B) the comparison of expression levels of CXCR2 in neutrophils isolated from the bone marrow of WT or PLD2 knockout (KO) mice after being stimulated with LPS, (C) the comparison of the change of CXCR2-induced neutrophil chemotaxis after neutrophils isolated from the bone marrow of WT or PLD2 knockout (KO) mice are stimulated with LPS, and (D) the comparison of the change of CXCR2-induced neutrophil chemotaxis when neutrophils isolated from the bone marrow of WT mice are treated with a vehicle or a PLD2 inhibitor and stimulated with LPS;

FIG. 18 shows (A) the comparison of GRK2 levels after neutrophils isolated from the bone marrow of WT or PLD2 knockout (KO) mice are stimulated with LPS, and (B) the comparison of GRK2 levels when neutrophils isolated from the bone marrow of WT mice are treated with a vehicle or a PLD2 inhibitor and stimulated with LPS;

FIG. 19 shows the comparison of GRK2 levels when neutrophils isolated from the bone marrow of WT mice are treated with an inhibitor (BAY11-7082) of an intracellular signaling pathway and stimulated with LPS;

FIG. 20 shows (A) the comparison of CXCR2 expression levels and (B) the comparison of the change of CXCR2-induced neutrophil chemotaxis, when neutrophils isolated from the bone marrow of WT mice are treated with a vehicle or BAY11-7082 and stimulated with LPS;

FIG. 21 shows the comparison of the variations of survival rates over time, after WT and PLD2 knockout (KO) mice subjected to CLP surgery are treated with CXCR2 antagonist SB225002;

FIG. 22 shows the results obtained by measuring a p65 translocation after neutrophils isolated from the bone marrow of WT or PLD2 knockout (KO) mice are stimulated with LPS;

FIG. 23 shows the comparison of bacterial colony counts in the peritoneal fluid isolated from a WT recipient mouse to which neutrophils of WT or PLD2 knockout (KO) mice (donor) are adoptive-transferred;

FIG. 24 shows the comparison of (A) lung inflammation and (B) spleen cell apoptosis, where the lung and the cells are isolated from a WT recipient mouse to which neutrophils of WT or PLD2 knockout (KO) mice (donor) are adoptive-transferred;

FIG. 25 shows the comparison of variations of inflammatory cytokine/chemokine expression in (A) the peritoneal fluid and (B) the bronchoalveolar lavage fluid (BALF), which are isolated from a WT recipient mouse to which neutrophils of WT or PLD2 knockout (KO) mice (donor) are adoptive-transferred;

FIG. 26 shows the in vivo trafficking ability of neutrophils in a WT recipient mouse to which CFSE-labeled neutrophils of WT or PLD2 knockout (KO) mice (donor) are adoptive-transferred, measured using a flow cytometer; and

FIG. 27 shows the comparison of the variations of survival rates over time, after a CLP surgery is performed on a WT recipient mouse to which CFSE-labeled neutrophils of WT or PLD2 knockout (KO) mice (donor) are adoptive-transferred.

### DETAILED DESCRIPTION OF EXEMPLARY EMBODIMENTS

Hereinafter, the present invention will be described in detail.

The present invention provides a pharmaceutical composition for preventing or treating a bacterial infectious disease, comprising a PLD2 inhibitor as an active ingredient.

The term “prevention” used herein refers to all of the actions for inhibiting or delaying the occurrence of a bacterial infectious disease by the administration of the composition of the present invention.

The term “treatment” used herein refers to all of the actions for relieving or beneficially changing the symptoms of a bacterial infectious disease by the administration of the composition of the present invention.

The term “bacterial infectious disease” used herein is a disease caused by bacteria (germs), and is preferably sepsis or septic shock, but the present invention is not limited thereto.

The present invention first identified the fact that PLD2 plays an important role in regulating the migration and activity of neutrophils in the pathological progression of a bacterial infectious disease, particularly, sepsis. It was first identified that, when PLD2 is inhibited (including knockout), the activity of peptidylarginine deiminase 4 (PAD4) in the neutrophils is increased and thus bactericidal activity induced by the formation of a neutrophil extracellular trap (NET) is enhanced, resulting in inhibition of immune cell apoptosis and neutrophil recruitment, and therefore, PLD2 inhibition is effective in treating sepsis or septic shock.

More particularly, in one exemplary embodiment of the present invention, it was confirmed that, when PLD2 is inhibited (including knockout), mortality caused by sepsis is considerably reduced (refer to Example 2), and lung inflammation and liver inflammation, which are associated with the mortality caused by sepsis, are also considerably reduced (refer to Example 3 and Example 4).

In another exemplary embodiment of the present invention, it was confirmed that, when PLD2 is inhibited (including knockout), production of inflammatory cytokines/chemokines is significantly reduced (refer to Example 5), and immune cell apoptosis is inhibited (refer to Example 6).

In still another exemplary embodiment of the present invention, it was confirmed that, when PLD2 is inhibited (including knockout), activation-dependent calcium signaling in neutrophils is improved, and calcium-dependent PAD enzyme activity is increased, resulting in histone 3 citrullination and NET formation in the neutrophils (refer to Example 7).

In yet another exemplary embodiment of the present invention, since PLD2 is necessary for activity of a main transcription factor for GRK2 expression (NF-κB) due to LPS in mouse neutrophils, resulting in regulation of the expression of GRK2, it was confirmed that PLD2 plays an important role in regulating the surface expression of CXCR2 (refer to Example 8).

For these reasons, a substance for suppressing or inhibiting PLD2 may be useful in preventing, relieving or treating a bacterial infectious disease, particularly, sepsis or septic shock.

A PLD2 inhibitor as an active ingredient in the composition of the present invention may be any substance that can inhibit PLD2, which is, but not particularly limited to, preferably, 5-fluoro-2-indolyl des-chlorohalopemide, N-[2-[1-(3-Fluorophenyl)-4-oxo-1,3,-8-triazaspiro[4.5]dec-8-yl]ethyl]-2-naphthalenecarboxamide, N-{2-[4-oxo-1-phenyl-1,3,8-triazaspiro(4.5)decan-8-yl]ethyl}quinoline-3-carboxamide, N-(2-{4-[2-oxo-2,3-dihydro-1H-benzo(d)imidazol-1-yl]piperidin-1-yl}ethyl)-2-naphthamide, (1R,2R)—N—([S]-1-{4-[5-bromo-2-oxo-2,3-dihydro-1H-benzo(d)imidazol-1-yl]piperidin-1-yl}propan-2-yl)-2-phenylcyclopropanecarboxamide), N-[2-[4-(5-chloro-2,3-dihydro-2-oxo-1H-benzimidazol-1-yl)-1-piperidinyl]-ethyl]-4-fluoro-benzamide, and more preferably, N-[2-(4-oxo-1-phenyl-1,3,8-triazaspiro[4,5]decan-8-yl)ethyl]-2-naphthalenecarboxamide.

In the pharmaceutical composition according to the present invention, the PLD2 inhibitor may be comprised at 0.1 to 60 wt % with respect to the total weight of the composition, but the present invention is not limited thereto.

The pharmaceutical composition according to the present invention may comprise a pharmaceutically effective amount of a PLD2 inhibitor alone, or in combination with one or more pharmaceutically acceptable carriers, excipients or diluents. Here, the term “pharmaceutically effective amount” refers to the amount sufficient to prevent or treat the symptoms of a bacterial infectious disease, particularly, sepsis or sepsis shock.

The pharmaceutically effective amount of the PLD2 inhibitor according to the present invention may be 0.5˜100 mg/day/kg (body weight), preferably, 1˜50 mg/day/kg (body weight), and more preferably, 10˜20 mg/day/kg (body weight). However, the pharmaceutically effective amount may be suitably changed depending on the severity of a symptom, a patient's age, body weight, health condition or sex, an administration route and the duration of treatment, but the present invention is not limited thereto.

Also, the term “pharmaceutically acceptable composition” refers to a composition which does not conventionally induce allergic responses such as gastroenteric disorders and dizziness or similar responses thereof, when the composition is physiologically acceptable and administered to a human. Examples of a carrier, excipient and diluent comprised in the composition may comprise lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinyl pyrrolidone, water, methyl hydroxybenzoate, propyl hydroxybenzoate, talc, magnesium stearate, mineral oil, etc. Also, the examples of a carrier, excipient and diluent comprised in the composition may further include a filler, an anti-coagulant, a lubricant, a wetting agent, a flavoring agent, an emulsifier, a preservative, etc.

Also, the composition of the present invention may be prepared in a dosage form by a method known in the art to provide a fast, sustained or delayed release of an active ingredient after being administered to a subject having a bacterial infectious disease, particularly, sepsis or sepsis shock, including a human. The dosage form may be, but is not limited to, a powder, a granule, a tablet, an emulsion, a syrup, an aerosol, a soft or hard gelatin capsule, a sterilized injection solution or sterilized powder.

The pharmaceutical composition according to the present invention may be administered through various routes including oral, percutaneous, subcutaneous, intravenous and intramuscular administration, and a dosage of the active ingredient may be suitably selected depending on various parameters such as an administration route, a patient's age, sex, body weight and severity of disease.

Also, the composition of the present invention may be used in combination with a known method such as a surgery, hormone treatment, drug treatment or a biological reaction regulator, which has an effect of preventing, relieving or treating the symptoms of a bacterial infectious disease, particularly, sepsis or sepsis shock. Particularly, for an infectious disease such as sepsis or septic shock, basically, an antibiotic is administered, and thus when the composition of the present invention is administered in combination with the antibiotic, the effect of the antibiotic may be reinforced. The antibiotic may be, for example, a β-lactam-based antibiotic, an aminoglycoside-based antibiotic, a macrolide-based antibiotic, a lincosamide-based antibiotic, a nitroimidazole-based antibiotic, a quinolone-based antibiotic, an ureidopenicillin-based antibiotic, or a glycopeptide-based antibiotic, which may be used alone or in combination thereof. Preferably, the antibiotic comprises one or a combination of ampicillin, azithromycin, benzyl penicillin, cefepime, ceftriaxone, cephazolin, clindamycin, flucloxacillin, gentamycin, cephalosporin, lincomycin, metronidazole, moxifloxacin, piperacillin, tazobactam, ticarcillin, clavulanic acid and vancomycin, but the present invention is not limited thereto.

In addition, the PLD2 inhibitor according to the present invention may be added to food for preventing or relieving a bacterial infectious disease, particularly, sepsis or sepsis shock, and also used as a health functional food composition to prevent or relieve the symptoms of sepsis or sepsis shock.

Therefore, the PLD2 inhibitor according to the present invention may be easily utilized to prepare health functional food, for example, a main ingredient, a supplementary ingredient, a food additive, a functional food or beverage, effective in preventing and relieving a bacterial infectious disease, particularly, sepsis or sepsis shock.

The term “health functional food” used herein refers to food designed and processed to sufficiently express body regulation functions involved with the regulation of a biological defense rhythm, disease prevention and recovery of the food group or food composition giving added values to act and express the function of corresponding food to be applicable for a specific purpose by processing food using a physical, biochemical or biotechnological technique, and may further include a nutritionally-acceptable supplementary food additive, and a proper carrier, excipient and diluent, which are conventionally used.

Also, the health functional food composition of the present invention may comprise a variety of nutrients, vitamins, minerals (electrolytes), flavors including synthetic and natural flavors, coloring agents and fillers (cheese, chocolate, etc.), pectic acid and a salt thereof, alginic acid and a salt thereof, an organic acid, a protective colloid thickening agent, a pH regulator, a stabilizer, a preservative, glycerin, alcohol, or a carbonating agent used for soft drinks, and the listed ingredient may be used alone or in combination with others.

An amount of the PLD2 inhibitor may be 0.001 to 90 wt %, and preferably, 0.1 to 40 wt % with respect to a total weight of the health functional food. When the health functional food is prepared for long term consumption, the PLD2 inhibitor may be comprised in the above range. However, since the active ingredient has no problem in terms of stability, the PLD2 inhibitor can be used in more than the above range, and thus the present invention is not limited thereto.

In yet another aspect, the present invention provides a method for treating a bacterial infectious disease, preferably, sepsis or septic shock by administering a pharmaceutically effective amount of a pharmaceutical composition comprising a PLD2 inhibitor as an active ingredient to a subject. The “subject” used herein refers to a target with a disease to be treated, more specifically a human, or a mammal such as a non-human primate, a mouse, a rat, a dog, a cat, a horse or a cow.

Hereinafter, exemplary examples will be provided to help in understanding of the present invention. However, the following examples are merely provided to more easily understand the present invention, but the scope of the present invention is not limited to the following examples.

### EXAMPLES

### Example 1

### Manufacture of Sepsis Mouse Models

1-1. CLP-Induced Sepsis Models

C57Bl/6 mice were obtained from Orient Bio (Seongnam, Korea). PLD2 knockout (KO) mice were prepared by the method described in a previous study (Ghim, J., J. S. Moon, C. S. Lee, J. Lee, P. Song, A. Lee, J. H. Jang, D. Kim, J. H. Yoon, Y. J. Koh, C. Chelakkot, B. J. Kang, J. M. Kim, K. L. Kim, Y. R. Yang, Y. Kim, S. H. Kim, D. Hwang, P. G. Suh, G. Y. Koh, Y. Y. Kong, and S. H. Ryu, 2014, “Endothelial deletion of phospholipase D2 reduces hypoxic response and pathological angiogenesis,” Arterioscler. Thromb. Vasc. Biol., 34:1697-1703.) All experiments involving animals received the approval of the Institutional Review Committee for Animal Care and Use at the medical school of Sungkyunkwan University. Cecal ligation and puncture (CLP) sepsis models were prepared by the method described in a previous study (Kim, S. D., H. Y. Lee, J. W. Shim, H. J. Kim, Y. H. Yoo, J. S. Park, S. H. Baek, B. A. Zabel, and Y. S. Bae, 2011, “Activation of CXCR2 by extracellular matrix degradation product acetylated Pro-Gly-Pro has therapeutic effects against sepsis,” Am. J. Respir. Crit. Care Med., 184:243-251.) That is, mice were anesthetized with 50 mg/kg of Zoletil and 10 mg/kg of Rompun through intraperitoneal injection, and an abdominal midline incision was made to expose the cecum. Subsequently, the cecum was ligated at the end of the ileocecal valve, punctured once or twice to measure cytokine production through a surface using a 22-gauge needle, and then the abdomen was sutured.

Meanwhile, sham CLP mice as a control group were subjected to the same method as described above, but the cecum was not punctured.

1-2. S. aureus-Induced Sepsis Models

S. aureus (2×10⁸ cells per mouse) were injected into peritoneal cavities of C57Bl/6 and PLD2 knockout (KO) mice.

### Example 2

### Confirmation of Variations of Survival Rates According to PLD2 Deficiency in Sepsis Models

2-1. Confirmation of Variations of Survival Rates in CLP-Induced Sepsis Models

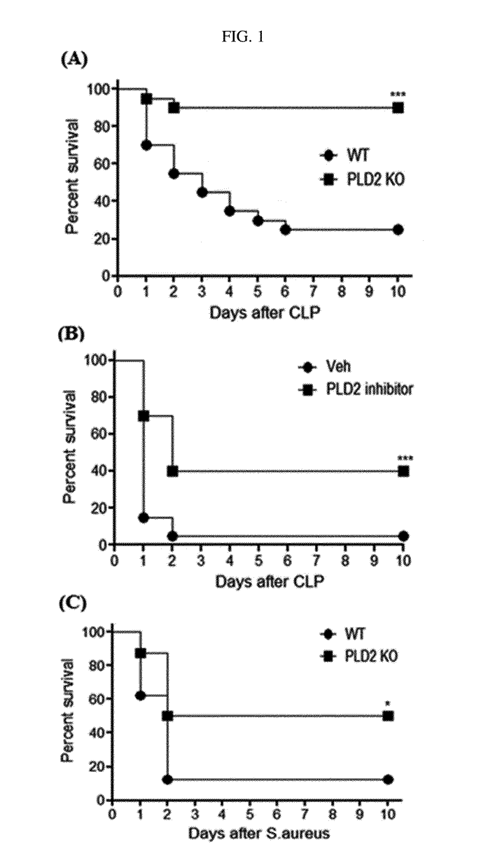

Wild-type (WT) and PLD2 knockout (KO) mice were subjected to CLP surgery as described in Example 1-1, and then the variations of survival rates over time were examined. As a result, as shown in FIG. 1A, it was confirmed that, on day 10 after the CLP surgery, the survival rate of the WT mice was only 20%, but the survival rate of the PLD2 knockout (KO) mice was 90%.

Additionally, at 2, 14, 26 and 38 hours after CLP surgery for the WT mice, the CLP mice were treated with a vehicle (0.5% Tween 80 in PBS) or 10 mg/kg of a PLD2 inhibitor ((N-[2-(4-oxo-1-phenyl-1,3,8-triazaspiro[4,5]decan-8-yl)ethyl]-2-naphthalenecarboxamide; CAY10594) four times through subcutaneous injection, and survival of each of the mice was monitored daily for 10 days. As a result, as shown in FIG. 1B, it was confirmed that the survival rate of the WT mice treated with the PLD2 inhibitor was 40%, which shows that the PLD2 inhibitor considerably prolonged the survival of the WT mice.

2-2. Confirmation of Variations of Survival Rates in S. aureus-Induced Sepsis Models

S. aureus is Gram-positive bacteria, which are the main cause of sepsis. S. aureus was injected into WT and PLD2 knockout (KO) mice by the same method as described in Example 1-2, and the variations of survival rates were examined. As a result, as shown in FIG. 1C, it was confirmed that, on day 10 after CLP surgery, the survival rate of the WT mice was only 10%, but the survival rate of the PLD2 knockout (KO) mice was 50%.

From the above results, it can be seen that PLD2, more specifically, PLD2 activity was involved in the pathological progression of sepsis, and thus PLD2 deficiency significantly reduced mortality in CLP-induced or S. aureus-induced sepsis.

### Example 3

### Confirmation of Relationship Between PLD2 Deficiency and Lung Inflammation in CLP-Induced Sepsis Models

3-1. Histological Observation

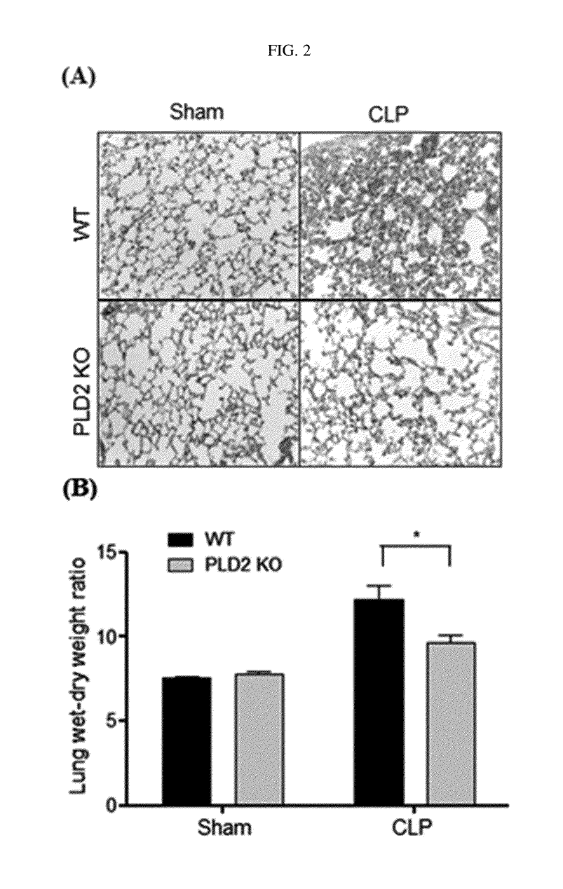

The close relationship between the mortality of CLP-induced sepsis mice and lung inflammation is well known (Cohen, J., 2002, “The immunopathogenesis of sepsis,” Nature, 420:885-891.) Here, WT and PLD2 knockout (KO) mice were subjected to sham or CLP by the same method as described in Example 1-1. At 24 hours after the surgery, the mice were euthanized, and the lung of a mouse was fixed, sectioned and then stained with hematoxylin and eosin for morphological analysis. As a result, as shown in FIG. 2A, it can be confirmed that, CLP caused acute inflammation of the lung accompanied with severe alveolar congestion and large-scale thrombosis in the WT mice, but did not cause such inflammation in the PLD2 knockout (KO) mice.

3-2. Quantification of Pulmonary Edema

To quantify the lung inflammation, the wet to dry (W/D) weight ratios of the lungs extracted from the WT and PLD2 knockout (KO) mice, which had been subjected to sham or CLP surgery by the same method as described in Example 1-1, were measured. That is, the mice were euthanized at 24 hours after the surgery, and the total weights of extracted wet lungs were measured and then placed in an oven at 60° C. for 48 hours. Subsequently, a dry weight was measured to estimate the W/D ratio. As a result, as shown in FIG. 2B, it can be confirmed that CLP surgery considerably increased the W/D ratio of the lung in the WT mice, which is evidence of edema. However, in the PLD2 knockout (KO) mice, no significant increase in the W/D ratio was observed.

### Example 4

### Confirmation of Relationship Between PLD2 Deficiency and Liver Inflammation in CLP-Induced Sepsis Models

4-1. Histological Observation

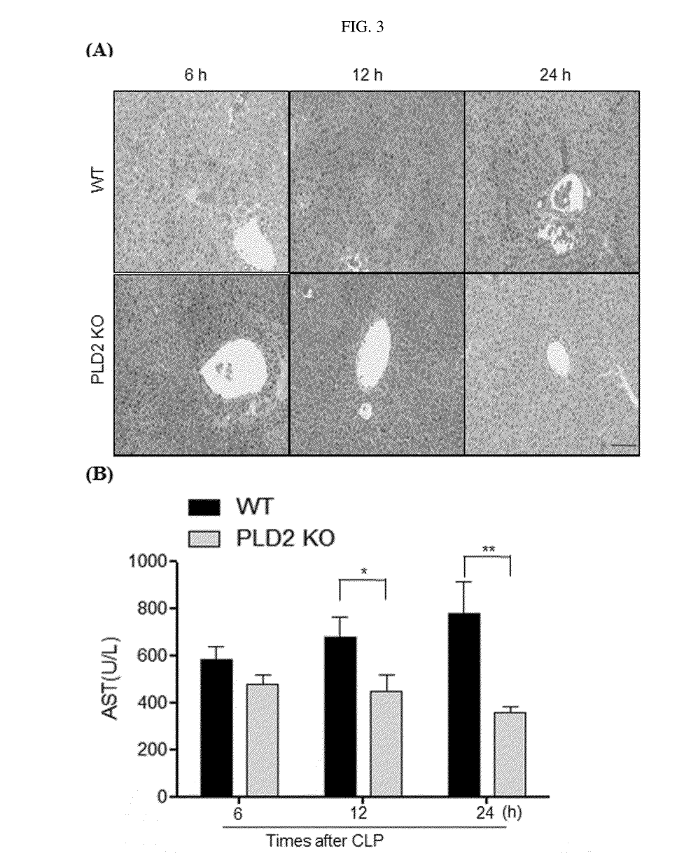

It is well known that the death caused by sepsis is associated with the dysfunction of main organs including the liver (Cohen, J. 2002. The immunopathogenesis of sepsis. Nature. 420:885-891). Accordingly, WT and PLD2 knockout (KO) mice were subjected to CLP surgery by the same method as described in Example 1-1. At 24 hours after the surgery, the mice were euthanized, and the liver thereof were fixed, sectioned and then stained with hematoxylin and eosin for morphological analysis. As a result, as shown in FIG. 3A, it can be confirmed that the CLP surgery caused apparent liver inflammation in the WT mice. Particularly, it can be confirmed that a serious liver damage was time-dependently generated at 24 hours after the CLP surgery. However, it can be confirmed that CLP-induced liver inflammation was considerably reduced in the PLD2 knockout (KO) mice.

4-2. Measurement of AST Level

A high plasma aspartate aminotransferase (AST) level is a marker for liver damage (Futter, L. E., O. A. al-Swayeh, and P. K. Moore, 2001, “A comparison of the effect of nitroparacetamol and paracetamol on liver injury,” Br. J. Pharmacol, 132:10-12.) Here, WT and PLD2 knockout (KO) mice were subjected to CLP surgery, and an AST level over time was measured using a commercially available kit (Sigma-Aldrich, St. Louis, Mo.) according to a standard laboratory technique. As a result, as shown in FIG. 3B, it can be confirmed that, compared with the PLD2 knockout (KO) mice, the plasma AST level was considerably increased in the WT mice after 12 and 24 hours.

### Example 5

### Confirmation of Variations of Inflammatory Cytokine/Chemokine Production According to PLD2 Deficiency

5-1. Confirmation of Variations of CLP-Induced Inflammatory Cytokine/Chemokine Production

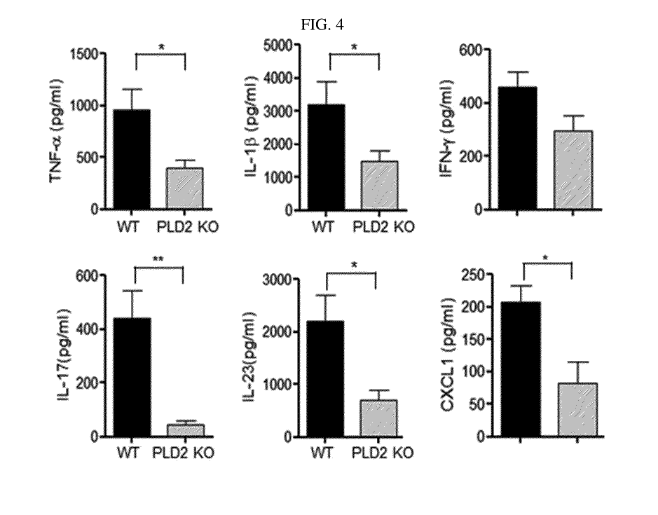

WT and PLD2 knockout (KO) mice were subjected to CLP surgery by the same method as described in Example 1-1. At 24 hours after the surgery, peritoneal lavage fluids were collected, the expression of cytokines and chemokines was measured in peritoneal fluids by ELISA (e-Bioscience, San Diego, Calif.). As a result, as shown in FIG. 4, it can be confirmed that, for first 24 hours, the CLP surgery considerably increased cytokines (TNF-α, IL-1β, IFN-γ, IL-17, and IL-23) and a chemokine (CXCL1) in peritoneal exudates of the WT mice, but the levels of such proinflammatory cytokines and chemokine were considerably decreased in peritoneal exudates of the PLD2 knockout (KO) mice.

5-2. Confirmation of Variations of LPS-Induced Inflammatory Cytokine/Chemokine Production

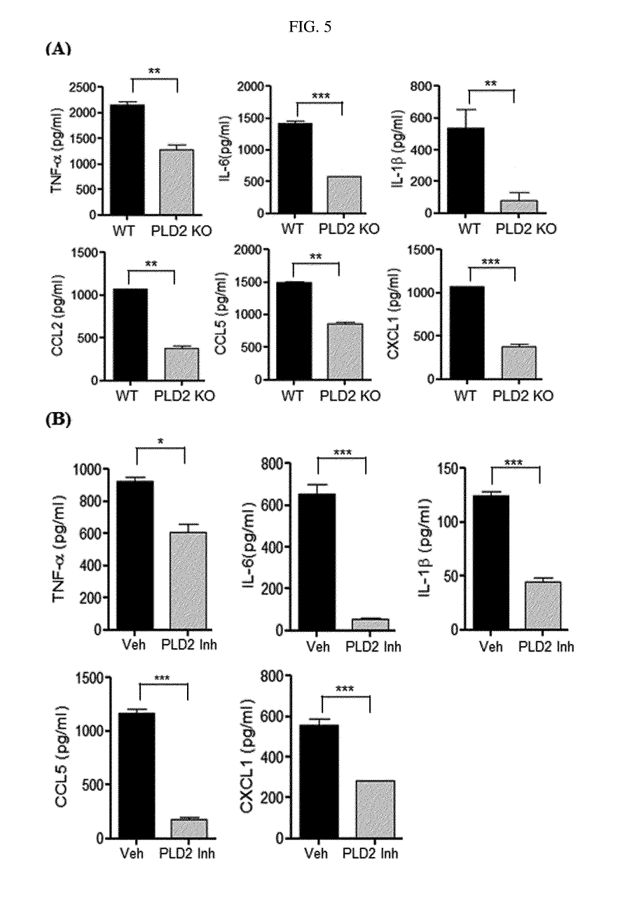

Spleen cells (3×10⁶ cells/0.3 ml) isolated from WT or PLD2 knockout (KO) mice were cultured in RPMI 1640 medium containing 5% fetal bovine serum (FBS) in 96-well plates and placed in a 5% CO₂ incubator at 37° C. Afterward, the spleen cells were incubated with LPS (1 μg/ml) for 24 hours, and the expression of cytokines and chemokines in the spleen cells was measured by ELISA (e-Bioscience, San Diego, Calif.). As a result, as shown in FIG. 5A, it can be confirmed that, compared with the WT mice, TNF-α, IL-6 and IL-1β secretion was considerably decreased in the spleen cells in the LPS-treated PLD2 knockout (KO) mice at 24 hours. Also, it can be confirmed that, compared with the WT mice, CCL2, CCL5 and CXCL1 secretion was considerably decreased in the spleen cells of PLD2 knockout (KO) mice stimulated with LPS.

Additionally, to examine the effect of a PLD2 inhibitor on cytokine and chemokine production, before additional culturing with LPS (1 μg/ml) for 24 hours, the spleen cells isolated from the WT mice were cultured with 10 μM of a vehicle (0.5% Tween 80 in PBS) or a PLD2 inhibitor (CAY10594) for 30 minutes. A cell-free supernatant was collected and centrifuged, and the cytokines and the chemokines were detected by ELISA (e-Bioscience, San Diego, Calif.) according to the manufacturer's instructions. As a result, as shown in FIG. 5B, it can be confirmed that, compared with the control treated with the vehicle, the TNF-α, IL-6, IL-1β, CCL5, and CXCL1 secretion was considerably decreased in the spleen cells of the WT mice treated with LPS and the PLD2 inhibitor.

### Example 6

### Confirmation of Inhibition of Apoptosis According to PLD2 Deficiency

Immune cell apoptosis is associated with the mortality by sepsis (Cohen, J. 2002. The immunopathogenesis of sepsis. Nature. 420:885-891). Here, to confirm the relationship between PLD2 deficiency and immune cell apoptosis, an experiment was performed as follows.

6-1. TUNEL Assay

WT or PLD2 knockout (KO) mice were subjected to sham or CLP surgery by the same method as described in Example 1-1. At 24 hours after the surgery, the mice were euthanized, and the spleen, thymus, kidney and liver were isolated. A TUNEL assay was performed on frozen tissue sections using a standard histological protocol. That is, the sections were permeabilized with Triton X-100 at 4° C. for 2 minutes, and immersed in a TUNEL reagent at 37° C. for 60 minutes. The percentage of apoptotic cells (TUNEL positive cells) was determined by counting a total of 500 cells using an optical microscope.

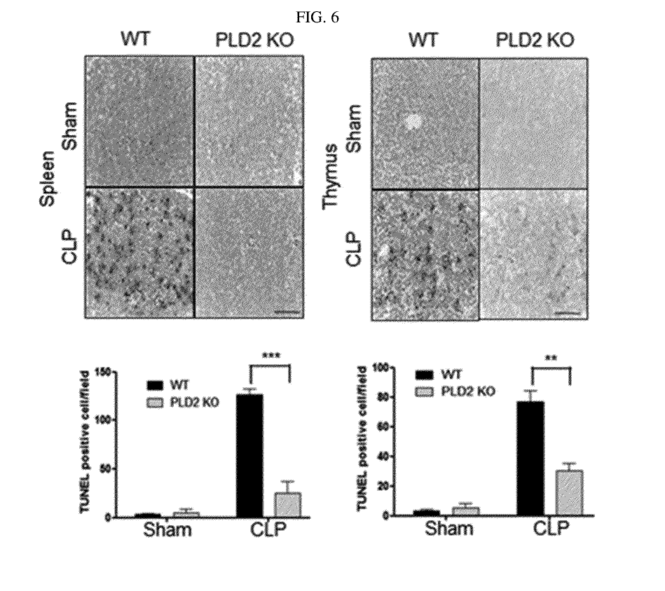

As shown in FIG. 6, it can be confirmed that the CLP surgery strongly induced the apoptosis of lymphocytes in the spleen and thymus. Also, it can be confirmed that a proportion of the TUNEL-positive cells was considerably decreased in the PLD2 knockout (KO) mice on which CLP surgery had been performed, compared with the WT mice on which CLP surgery had been performed. The above results show that PLD2 deficiency considerably reduced the apoptosis of lymphocytes in the spleen and thymus, resulting in the prevention of apoptosis induced by sepsis.

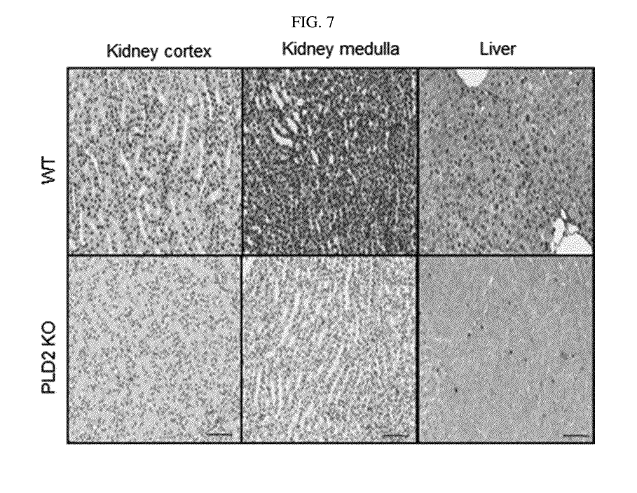

Also, as shown in FIG. 7, it can be confirmed that the CLP surgery considerably induced the apoptosis of renal cells in the kidney cortex and kidney medulla of the WT mice, but significantly reduced the apoptosis of renal cells in the kidney of the PLD2 knockout (KO) mice.

Moreover, it can also be confirmed that, as shown in FIG. 7, compared with the PLD2 knockout (KO) mice, apoptotic cells in the liver were considerably increased in the WT mice.

6-2. Flow Cytometry

WT or PLD2 knockout (KO) mice were subjected to sham or CLP surgery by the same method as described in Example 1-1. At 24 hours after the surgery, the mice were euthanized, and the spleens were isolated. The apoptosis of spleen cells was determined by Annexin-V/7AAD-positive staining. The Annexin-V/7AAD co-staining was performed using the Annexin-V-FITC/7AAD kit manufactured by Beckman Coulter, and analyzed using an FACSCanto II flow cytometer (BD Biosciences, San Jose, Calif.).

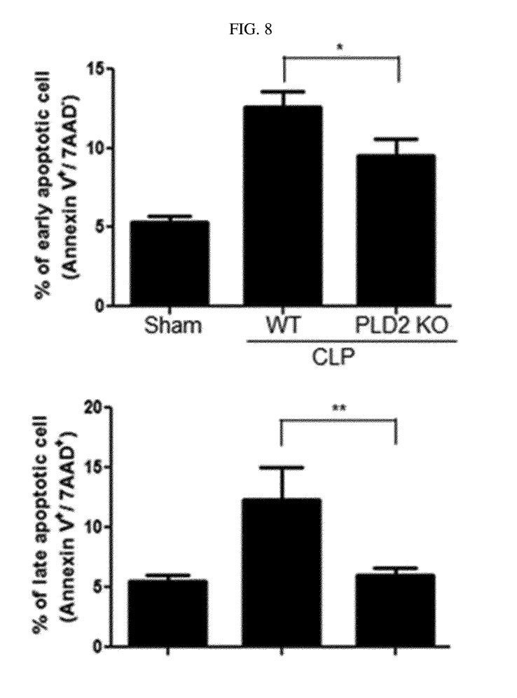

As a result, as shown in FIG. 8, it can be confirmed that the CLP increased the population of annexin V+/7AAD+ cells in the WT mice, but the population of annexin V+/7AAD+ cells were considerably decreased in the PLD2 knockout (KO) mice.

The above results show that, compared with the PLD2 knockout (KO) mice, the main organs are more seriously damaged in the WT mice, and PLD2 serves as a pathogenic contributor to apoptosis caused by sepsis.

### Example 7

### Confirmation of Bactericidal Activity According to PLD2 Deficiency

7-1. Confirmation of Variations of Bacterial Colony Counts

Apoptosis caused by sepsis is associated with bacterial colony counts in peritoneal fluid and peripheral blood (Xiao, H., J. Siddiqui, and D. G. Remick, 2006, “Mechanisms of mortality in early and late sepsis,” Infect. Immun., 74:5227-5235.) Here, to confirm the effect of bacterial clearance according to PLD2 deficiency in WT and PLD2 knockout (KO) CLP mice, an experiment was performed as follows.

WT or PLD2 knockout (KO) mice were subjected to sham or CLP surgery by the same method as described in Example 1-1. At 24 hours after the surgery, the peritoneal lavage fluids, peripheral blood and the bronchoalveolar lavage fluids (BALFs) were collected, and cells were cultured overnight at 37° C. in blood-agar base plates (Trypticase Soy Agar Deeps; Becton Dickinson, Franklin Lakes, N.J., USA). The lungs, the livers and the spleens were also isolated, and 10 mg of tissue of each organ were homogenized in 700 μL of PBS. Afterward, 50 μL of a tissue homogenate was cultured overnight at 37° C. in blood-agar plates. Colony forming units (CFUs) were determined as described in a previous study (Kim, S. D., H. Y. Lee, J. W. Shim, H. J. Kim, Y. H. Yoo, J. S. Park, S. H. Baek, B. A. Zabel, and Y. S. Bae, 2011, “Activation of CXCR2 by extracellular matrix degradation product acetylated Pro-Gly-Pro has therapeutic effects against sepsis,” Am. J. Respir. Crit. Care Med., 184:243-251.)

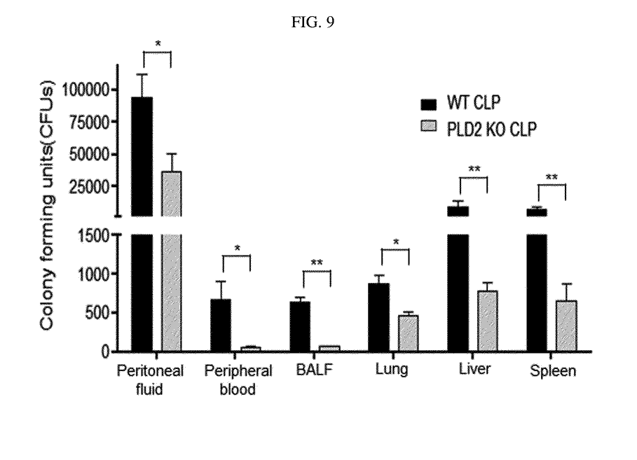

As a result, as shown in FIG. 9, it can be confirmed that, compared with the WT mice, a bacterial colony count was significantly decreased to about 60% in the peritoneal lavage fluid of the PLD2 knockout (KO) mice at 24 hours after the CLP. Also, compared to the WT mice, bacterial colony counts were considerably decreased even in the peripheral blood and the bronchoalveolar lavage fluid (BALF) of the PLD2 knockout (KO) mice.

Moreover, bacteria permeated into the abdominal cavity were finally permeated into lung tissues through circulation, and thus lung inflammation occurred (Matute-Bello, G., C. W. Frevert, O. Kajikawa, S. J. Skerrett, R. B. Goodman, D. R. Park, and T. R. Martin, 2001, “Septic shock and acute lung injury in rabbits with peritonitis: failure of the neutrophil response to localized infection,” Am. J. Respir. Crit. Care Med., 163:234-243.) Also, as shown in FIG. 9, it can be confirmed that the bacterial colony counts in lung, liver and spleen tissue considerably decreased in the PLD2 knockout (KO) mice, compared with the WT mice.

7-2. Confirmation of NET Formation

Recently, it has been reported that neutrophils form an NET to trap and kill invading bacteria (Cohen, V., U. Reichard, C. Goosmann, B. Fauler, Y. Uhlemann, D. S. Weiss, Y. Weinrauch, and A. Zychlinsky, 2004, “Neutrophil extracellular traps kill bacteria,” Science, 303:1532-1535.) Here, to examine the effect of PLD2 deficiency on NET formation, an experiment was performed as follows.

Neutrophils were isolated from the bone marrow of the WT or PLD2 knockout (KO) mice. More particularly, bone marrow cells were isolated from a femur and a tibia using an HBSS-EDTA solvent. A cell suspension was centrifuged at 400 g for 10 minutes, resuspended cells were loaded onto 52%, 69%, and 78% Percoll gradients, and centrifuged at 1500 g for 30 minutes without breaking. The cells were isolated on a 69%/78% interface layer, and RBCs were removed by hypotonic lysis. 95% or more of the isolated cells were identified to be Ly6G-positive using a flow cytometer. The isolated neutrophils (1×10⁵ cells) were seeded on 0.001% poly-lysine-coated 12-mm cover slips of 24-well plates, and stimulated with 5 μM ionomycin. Afterward, the cells were fixed with 4% paraformaldehyde and stained by SYTOX Green nucleic acid staining (5 μM). The NET was visualized using a Zeiss Axiovert fluorescent microscope (Zeiss, Oberkochen, Germany), and images were taken by a Nikon Digital camera.

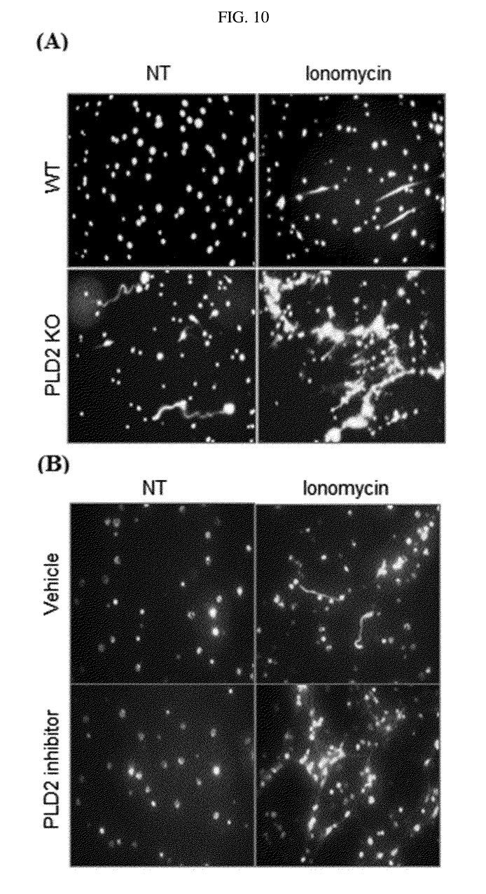

As a result, as shown in FIG. 10A, it can be confirmed that the neutrophils isolated from the ionomycin-stimulated WT mice have induced NET formation, and the NET formation was significantly increased in ionomycin-stimulated neutrophils isolated from the PLD2 knockout (KO) mice. Meanwhile, the NET formation, without stimulation with ionomycin, was slightly increased in the neutrophils isolated from the PLD2 knockout (KO) mice, compared with those isolated from the WT mice.

Additionally, to confirm the variations of NET formation when a PLD2 inhibitor was treated, neutrophils isolated from the bone marrow of the WT mice were cultured with 10 μM of a vehicle (0.5% Tween 80 in PBS) or a PLD2 inhibitor (CAY10594), and then stimulated with 5 μM ionomycin.

As a result, as shown in 10B, it can be confirmed that the NET formation caused by ionomycin stimulation was also increased in the neutrophils of the WT mice by the PLD2 inhibitor.

7-3. Confirmation of Histone 3 Citrullination

Histone citrullination is associated with NET formation (Wang, Y., M. Li, S. Stadler, S. Correll, P. Li, D. Wang, R. Hayama, L. Leonelli, H. Han, S. A. Grigoryev, C. D. Allis, and S. A. Coonrod, 2009, “Histone hypercitrullination mediates chromatin decondensation and neutrophil extracellular trap formation,” J. Cell Biol., 184:205-213.) Here, a citrullinated histone 3 level according to PLD2 deficiency was identified.

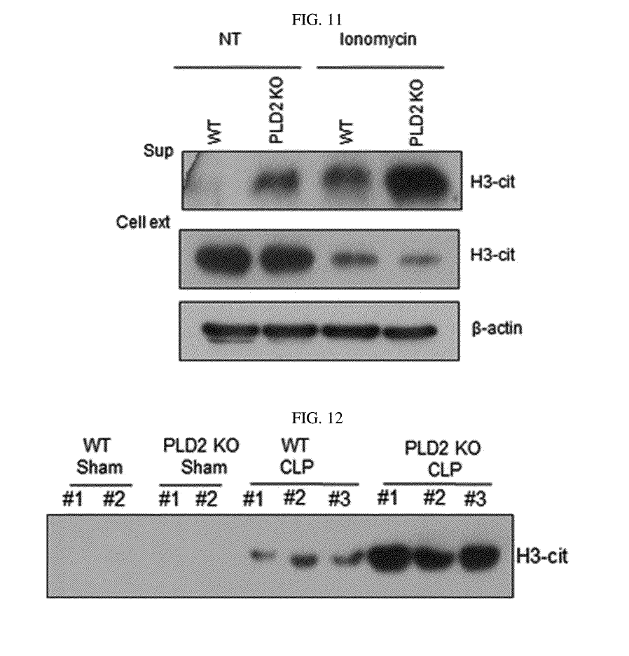

First, neutrophils were isolated from the bone marrow of WT or PLD2 knockout (KO) mice by the same method as described in Example 7-2, and stimulated with ionomycin, and then the level of citrullinated histone 3 in each neutrophil was measured. As a result, as shown in FIG. 11, it can be confirmed that, compared with the neutrophils isolated from the WT mice, the levels of citrullinated histone 3 in the neutrophils isolated from the PLD2 knockout (KO) mice were considerably increased. More particularly, it can be confirmed that, compared with neutrophils isolated from the PLD2 knockout (KO) mice and not stimulated with ionomycin or neutrophils isolated from the WT mice and stimulated with ionomycin, the levels of citrullinated histone 3 were considerably increased in the ionomycin-stimulated neutrophils isolated from the PLD2 knockout (KO) mice.

Subsequently, the WT or PLD2 knockout (KO) mice were subjected to sham or CLP surgery by the same method as described in Example 1-1. At 24 hours after the surgery, peritoneal fluids were collected, 1 volume of chloroform and 4 volumes of methanol were added, and secretion proteins were precipitated in a supernatant. The proteins were harvested at a liquid interface and dehydrated using speed-Vac. Afterward, the proteins were separated by SDS-PAGE and transferred onto a polyvinylidene difluoride membrane (Millipore). The level of citrullinated histone 3 was measured using anti-histone H3 (citrulline R2+R8+R17) antibody (Abcam 5103). As a result, as shown in FIG. 12, it was confirmed that, compared with the WT mice, the histone 3 citrullination level was considerably increased in peritoneal exudates of the PLD2 knockout (KO) mice after the CLP.

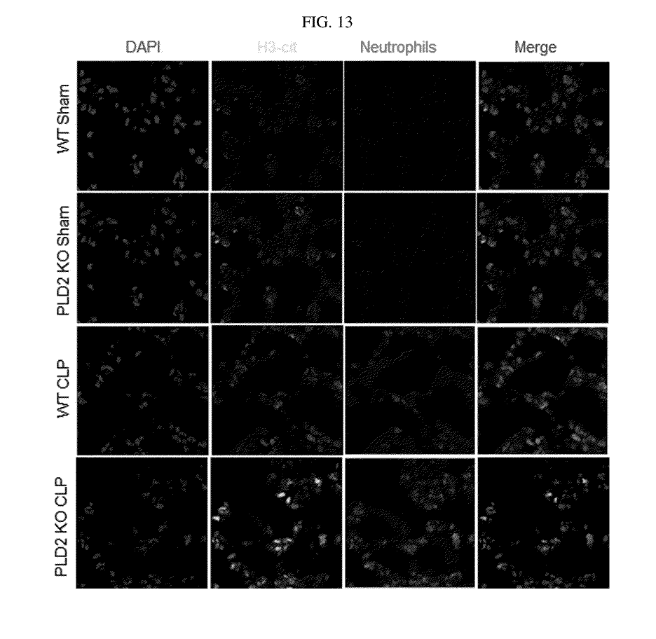

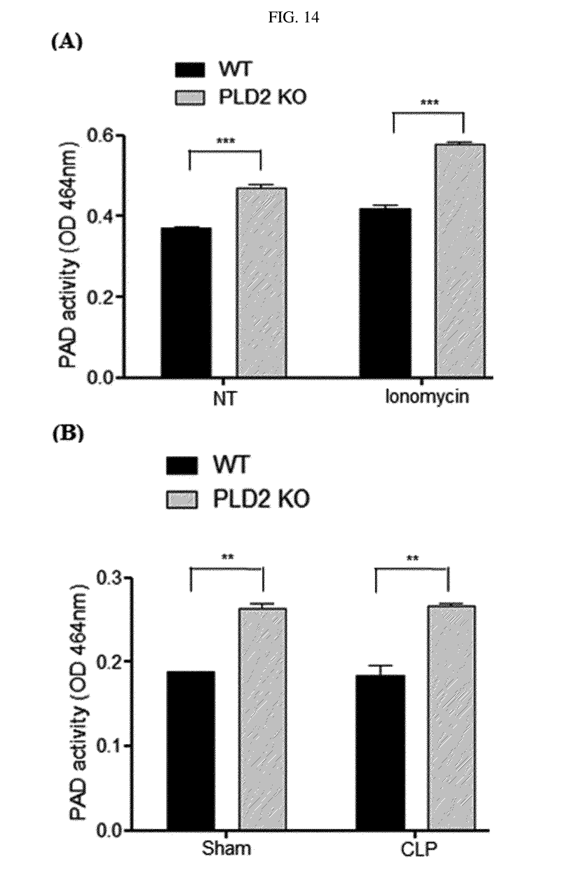

Subsequently, WT or PLD2 knockout (KO) mice were subjected to sham or CLP surgery by the same method as described in Example 1-1, and euthanized after 2 hours. An isolated lung was embedded in an OCT compound, and lung sections were fixed with methanol at −20° C. for 10 minutes. Each lung section was blocked with a blocking solution (PBS with 10% normal goat serum, 0.01% Triton X-100) at room temperature for 1 hour, neutrophils and citrullinated histone 3 were detected using rat anti-NIMP-R14 and rabbit anti-citrullinated histone H3 (citH3, Abcam). Here, anti-rat IgG AF594 and anti-rabbit IgG AF 488 (Invitrogen) were used as secondary antibodies. DNA was stained with DAPI (Santa Cruz), and placed on a glass slide. Cells were visualized using Zeiss LSM 500. As a result, as shown in FIG. 13, it can be confirmed that citrullinated histone 3 and NET formation were considerably increased in the lung neutrophils of the CLP-treated PLD2 knockout (KO) mice, compared with the WT mice.

7-4. Confirmation of Peptidylarginine Deiminase 4 (PAD4) Activity

Peptidylarginine deiminase 4 (PAD4) promotes histone 3 citrullination (Li, P., M. Li, M. R. Lindberg, M. J. Kennett, N. Xiong, and Y. Wang. 2010. “PAD4 is essential for antibacterial innate immunity mediated by neutrophil extracellular traps,” J. Exp. Med., 207:1853-1862.) It was confirmed that the histone 3 citrullination was upregulated in the neutrophils isolated from the PLD2 knockout (KO) mice by Example 7-3, and then PAD activity was measured in the corresponding neutrophils.

The PAD activity was measured by colorimetry for citrulline produced by PAD-stimulated citrullination of N-α-Benzoyl-L-arginine ethyl ester hydrochloride (BAEE). More particularly, 10 μg of a cell lysate or lung extract reacted with 5 mM BAEE at 55° C., and after 30 minutes, the reaction was stopped with 25 μl of 5M HClO₄, which was additionally added. Subsequently, a supernatant was mixed with reagent A (0.5% w/v diacetyl monoxime and 15% w/v NaCl in water) and reagent B (1% w/v antipyrine, 0.15% w/v ferric chloride, 25% v/v H₂SO₄ and 25% v/v H₃PO₄) for citrulline assay. The mixture was heated for 10 minutes and cooled in ice, and then the absorbance of the mixture was detected at 464 nm. As a result, as shown in FIG. 14, it was confirmed that the PAD activity was significantly increased in the PLD2 knockout (KO) neutrophils, compared with the WT (FIG. 14A). Also, compared with the WT mice, increased PAD activity was found in lung tissues of the PLD2 knockout (KO) mice as well as the sham PLD2 knockout (KO) mice 2 hours after the CLP (FIG. 14B).

7-5. Confirmation of Intracellular Calcium Mobilization

Intracellular calcium ions are required for the PAD4 activity, and induce histone 3 citrullination and NET formation (Rohrbach, A. S., D. J. Slade, P. R. Thompson, and K. A. Mowen, 2012, “Activation of PAD4 in NET formation,” Front. Immunol., 3:360.) Through Examples 7-2 and 7-3, it was confirmed that the ionomycin increased NET formation and histone 3 citrullination in the neutrophils of the PLD2 knockout (KO) mice, and then ionomycin-induced intracellular calcium mobilization was identified in the corresponding neutrophils.

The concentration of intracellular calcium ([Ca2+]i) was measured using Fura-2/AM according to Grynkiewicz's method, described in a previous study (Grynkiewicz, G., M. Poenie, and R. Y. Tsien, 1985, “A new generation of Ca2+ indicators with greatly improved fluorescence properties,” J. Biol. Chem., 260:3440-3450.) That is, the murine neutrophils isolated from the WT or PLD2 knockout (KO) mice were continuously stirred, and cultured at 37° C. for 50 minutes using 3 μM Fura-2/AM. Subsequently, for individual analysis, the cells were aliquoted (2×10⁶ cells/1 ml of Locke's solution containing 154 mM NaCl, 5.6 mM KCl, 1.2 mM MgCl₂, 5 mM HEPES [pH 7.3], 10 mM glucose, and 2 mM CaCl₂). Afterward, the cells were stimulated with 5 μM ionomycin. Meanwhile, to check the case of the treatment with a PLD2 inhibitor, the cells aliquoted from the WT mouse were cultured with 10 μM of a vehicle (0.5% Tween 80 in PBS) or a PLD2 inhibitor (CAY10594) before being stimulated with ionomycin. A fluorescence change at an emission wavelength of 500 nm was measured using an RF-5301PC spectrofluorophotometer (Shimadzu Instruments Inc., Kyoto, Japan) at excitation wavelengths of 340 nm and 380 nm A [Ca²⁺]ᵢ increase causes an increase in the fluorescence ratio at an excitation efficiency of 340 to 380 nm, and [Ca²⁺]ᵢ was calculated using the fluorescence ratio according to Equation 5 of Grynkiewicz et al (Grynkiewicz et al., 1985).

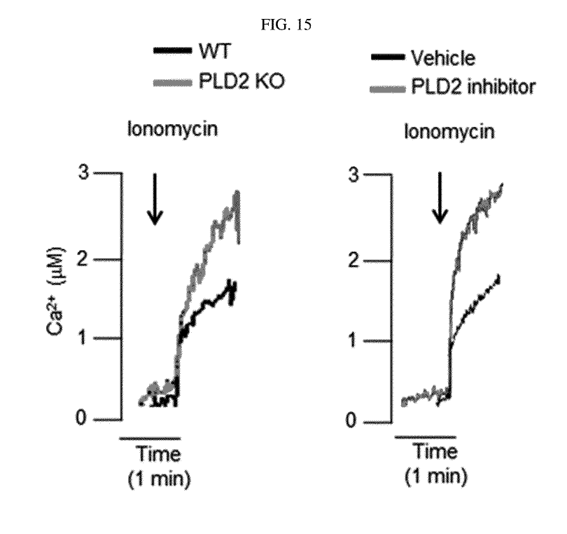

As a result, as shown in FIG. 15, it can be confirmed that, compared with the WT mice, an ionomycin-stimulated calcium flux was increased in the neutrophils of the PLD2 knockout (KO) mice. Also, the PLD2 inhibitor improved the ionomycin-induced intracellular calcium mobilization in the WT neutrophils.

The above results shows that PLD2 deficiency improved activation-dependent calcium signaling in the neutrophils, increased calcium-dependent PAD enzyme activity, leading to histone 3 citrullination and NET formation in the neutrophils.

### Example 8

### Confirmation of Induction of Neutrophil Recruitment According to PLD2 Deficiency

8-1. Evaluation of White Blood Cells

Lung inflammation may be generated by living bacteria in sepsis. The living bacteria released from CLP surgery migrates to the lungs through blood flow, and then, goes into the respiratory tract (Xiao, H., J. Siddiqui, and D. G. Remick, 2006, “Mechanisms of Mortality in Early and Late Sepsis,” Infect. Immun., 74:5227-5235.) Therefore, an experiment was performed to evaluate white blood cells in bronchoalveolar lavage fluids (BALFs) of the CLP mice as follows.

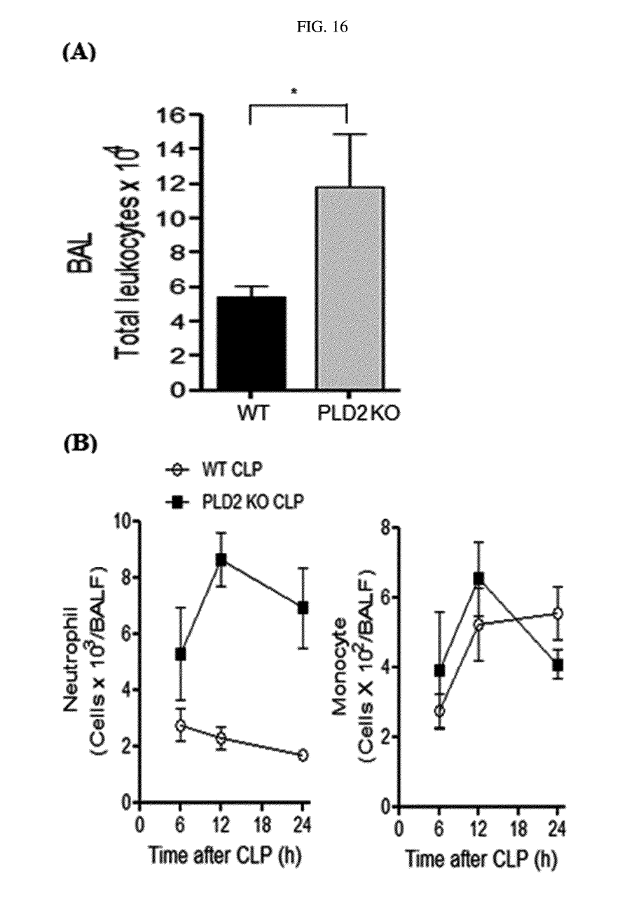

CLP surgery was performed on the WT or PLD2 knockout (KO) mice according to the method described in Example 1-1, and then the mice were euthanized 6, 12 or 24 hours after the surgery. To obtain the BALF, a cannula was inserted into the trachea after exsanguination, and the respiratory tract was washed with 900 μl PBS. The BALF sample was subjected to centrifugation (300 g, 10 min), thereby isolating cells. All cells obtained from the lung were stained with anti-CD11b (M1/70) antibody. Neutrophils or macrophages were stained with anti-Ly-6G (1A8) or anti-Ly6C (HK 1.4), respectively. The cells were analyzed using an FACSCanto II flow cytometer, and data was analyzed by FlowJo 7.6.5. As a result, as shown in FIG. 16, it can be confirmed that, in the PLD2 knockout (KO) mice, the total number of BALF white blood cells was considerably higher than the WT mice (FIG. 16A). Also, the neutrophils were strongly recruited into the respiratory tract in the PLD2 knockout (KO) mice, compared with the WT mice after the CLP. Meanwhile, there was no difference in numbers of BALF monocytes between the WT and the PLD2 knockout (KO) mice after the CLP (FIG. 16B).

8-2. Evaluation of CXCR2 Levels in Neutrophils

In a previous study, it was reported that a sepsis patient has lower CXCR2 expression on the surface of a neutrophil than a normal person (Cummings, C. J., T. R Martin, C. W. Frevert, J. M. Quan, V. A. Wong, S. M. Mongovin, T. R. Hagen, K. P. Steinberg, and R. B. Goodman. 1999. Expression and function of the chemokine receptors CXCR1 and CXCR2 in sepsis. J. Immunol. 162:2341-2346.), and a CXCR2 expression level is a key factor for controlling the recruitment of the neutrophils into an event area (Alves-Filho, J. C., F. Sônego, F. O. Souto, A. Freitas, W. A. Verri Jr, M. Auxiliadora-Martins, A. Basile-Filho, A. N. McKenzie, D. Xu, F. Q. Cunha, and F. Y. Liew, 2010, “Interleukin-33 attenuates sepsis by enhancing neutrophil influx to the site of infection,” Nat. Med., 16:708-712.) Therefore, the CXCR2 expression levels in neutrophils isolated from CLP-conducted WT or PLD2 knockout (KO) mice were identified through flow cytometry.

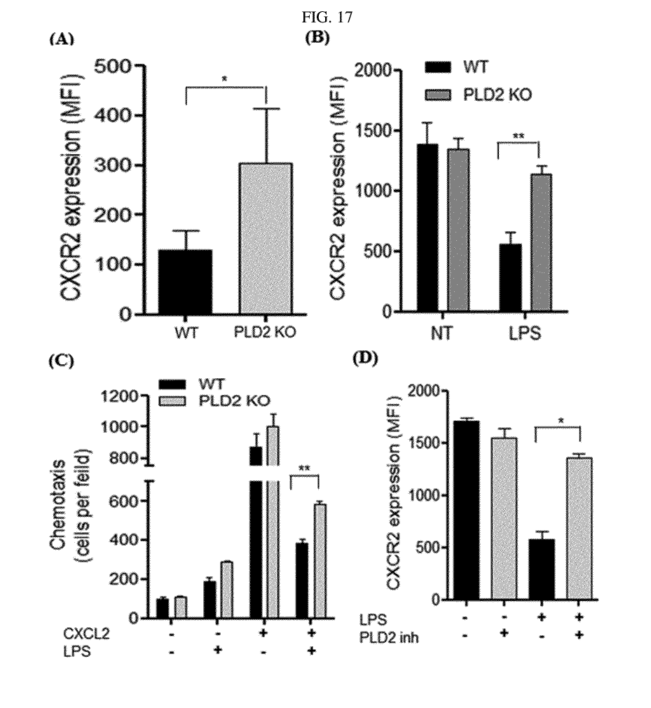

First, WT or PLD2 knockout (KO) mice were subjected to CLP surgery according to the method described in Example 1-1, and the mice were euthanized 6 hours after the surgery. Cells were collected from BALF, stained with antibodies, and analyzed by a flow cytometer: CD11b (M1/70) and Ly-6G (1A8) obtained from eBioscience (San Diego, Calif.), and CXCR2 (TG11/CXCR2) obtained from BioLegend (San Jose, Calif.). As a result, as shown in FIG. 17A, it can be confirmed that a CXCR2 expression level was considerably higher in neutrophils of the PLD2 knockout (KO) mice than the WT mice.

Afterward, neutrophils were isolated from the bone marrow of the WT or PLD2 knockout (KO) mice, stimulated with 1 μg/ml of LPS for 1 hour, stained, and analyzed using a flow cytometer. As a result, as shown in FIG. 17B, it can be confirmed that a surface expression level of CXCR2 in the WT neutrophils was significantly decreased by LPS stimulation. In addition, as shown in FIG. 17C, CXCR2-mediated neutrophil chemotaxis by CXCL2 was diminished by the LPS stimulation in the WT mice, and such an LPS-induced inhibitory effect caused the opposite result in the PLD2 knockout (KO) mouse. Meanwhile, it can be confirmed that the downregulation of LPS-induced CXCR2 was recovered in the WT neutrophils when a PLD2 inhibitor was treated (FIG. 17D).

8-3. Evaluation of GRK2 Level in Neutrophils

It was reported that the surface-expressed CXCR2 is phosphorylation-dependently downregulated, and GRK2 is used to phosphorylate CXCR2 (Angus, D. C., W. T. Linde-Zwirble, J. Lidicker, G. Clermont, J. Carcillo, and M. R. Pinsky. 2001. Epidemiology of severe sepsis in the United States: analysis of incidence, outcome, and associated costs of care. Crit. Care. Med. 29:1303-1310). Therefore, PLD2 deficiency in GRK2 expression induced by LPS was confirmed.

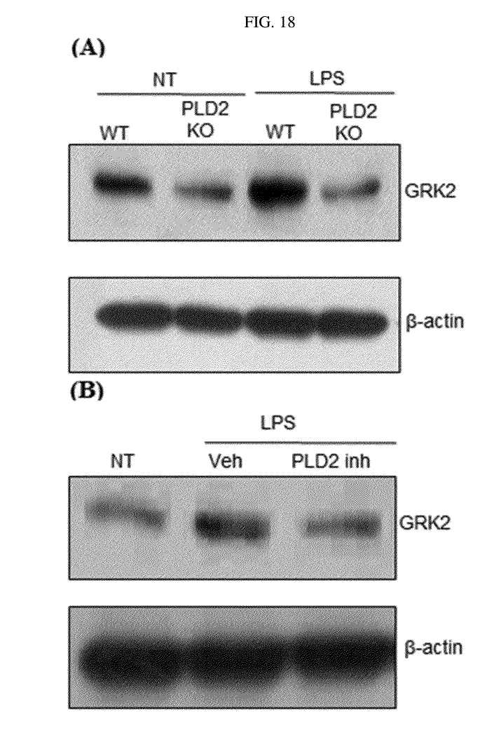

First, neutrophils were isolated from the bone marrow of WT or PLD2 knockout (KO) mice and stimulated with 1 μg/ml of LPS for 1 hour, and then GRK2 levels were analyzed by western blotting. As a result, as shown in FIG. 18A, it can be confirmed that, when the neutrophils of the WT mice were stimulated with LPS, the upregulation of GRK2 occurred, and when neutrophils isolated from the PLD2 knockout (KO) mice were stimulated with LPS, such upregulation of GRK2 was considerably diminished.

Afterward, neutrophils isolated from the bone marrow of the WT mice were cultured with 10 μM of a vehicle (0.5% Tween 80 in PBS) or a PLD2 inhibitor (CAY10594) before being cultured with LPS (1 μg/ml), and then GRK2 levels were analyzed by western blotting. As a result, as shown in FIG. 18B, it was confirmed that the upregulation of LPS-induced GRK2 was blocked.

The above results show that PLD2 plays a critical role in control of CXCR2 surface expression by controlling the expression of GRK2 in the murine neutrophils.

8-4. Confirmation of Signaling Pathway of LPS-Induced GRK2 Expression

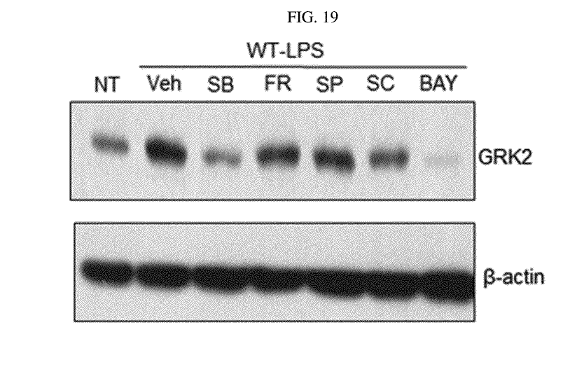

The control of LPS-induced GRK2 expression by an inhibitor for an intracellular signaling pathway (BAY11-7082) was analyzed by western blotting. As a result, as shown in FIG. 19, it can be confirmed that the LPS-induced GRK2 expression was completely inhibited by IκBα inhibitor BAY11-7082. The above result shows that the LPS-induced GRK2 expression was an NF-κB-dependent procedure.

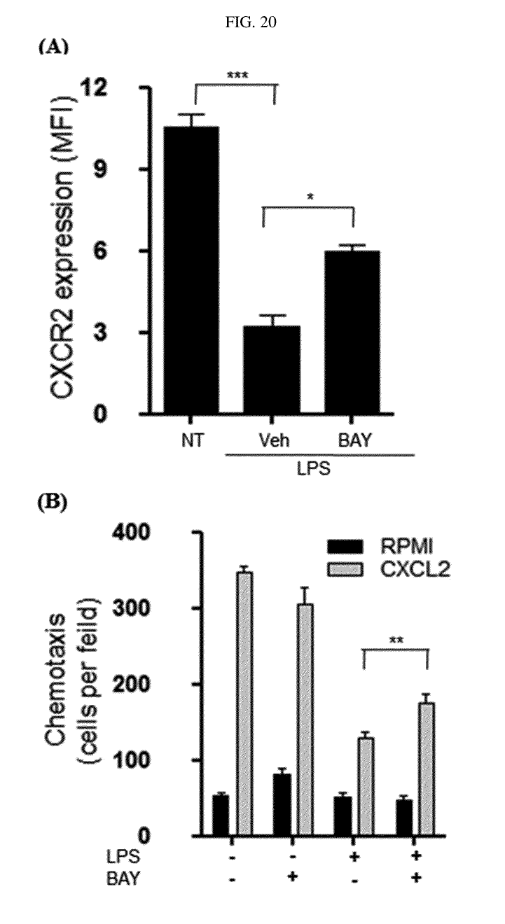

To verify the signaling pathway, additionally, neutrophils isolated from the bone marrow of WT mice were treated with a vehicle or BAY11-7082 and cultured with LPS (1 μg/ml), and then CXCR2 expression levels were identified. As a result, as shown in FIG. 20, it can be confirmed that the LPS-induced CXCR2 downregulation was significantly diminished by BAY11-7082 (FIG. 20A). Also, CXCL2-induced chemotactic migration of the neutrophils by LPS stimulation was partially but significantly recovered by the BAY11-7082 treatment (FIG. 20B).

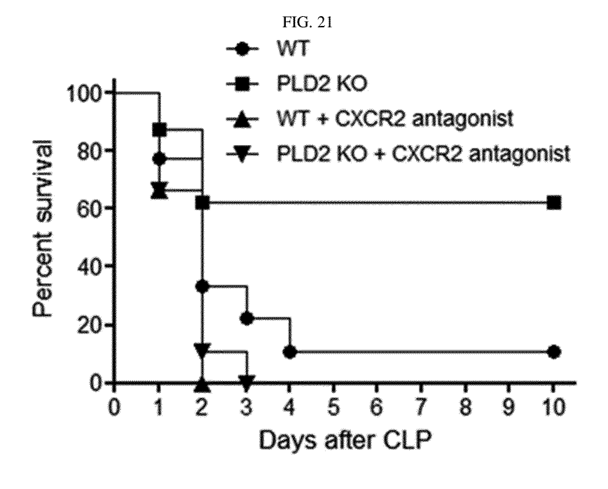

Also, WT or PLD2 knockout (KO) mice were subjected to CLP surgery according to the method described in Example 1-1 and treated with CXCR2 antagonist SB225002, and the variations of survival rates over time were identified. As a result, as shown in FIG. 21, it was confirmed that, in the PLD2 knockout (KO) mice, the survival rate maintained by PLD2 deficiency was not maintained any longer due to the treatment of the CXCR2 antagonist SB225002, and thus all of the mice died.

From the results, it can be seen that PLD2, more specifically, PLD2 activity is involved in the pathological progression of sepsis, and thus PLD2 deficiency significantly reduces mortality in CLP-induced or S. aureus-mediated sepsis.

8-5. Evaluation of p65 Nuclear Translocation in Neutrophils

NF-κB activity is associated with p65 nuclear translocation (Maguire, O., C. Collins, K. O'Loughlin, J. Miecznikowski, and H. Minderman, 2011, “Quantifying nuclear p65 as a parameter for NF-κB activation: Correlation between ImageStream cytometry, microscopy, and Western blot,” Cytometry A. 79:461-469.)

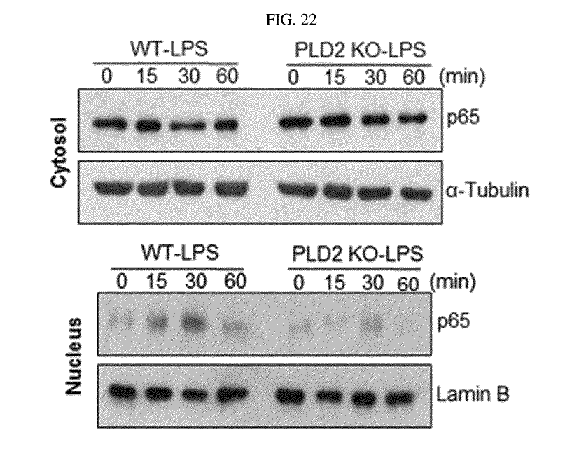

Here, neutrophils were isolated from the bone marrow of WT or PLD2 knockout (KO) mice, and cells were stimulated with LPS, and then p65 translocation was measured. More specifically, for subcellular fractionation, a subcellular protein fraction kit (Thermo, cat. 78840) was used. Cellular compartments were continuously extracted sequentially using a cytoplasmic extraction buffer (CEB) and a nuclear extraction buffer (NEB). Proteins were separated by SDS-PAGE and transferred onto a nitrocellulose membrane. Levels of p65, α-tubulin and lamin B were detected using antibodies for the respective proteins. As a result, as shown in FIG. 22, it can be confirmed that, when the neutrophils isolated from the WT mice were stimulated with LPS, p65 nuclear translocation was induced, but in the neutrophils of the PLD2 knockout (KO) mice, such p65 nuclear translocation was considerably diminished. The above results show that PLD2 is necessary for the activation of a principal transcription factor for GRK2 expression (NF-κB) by LPS in murine neutrophils.

### Example 9

### Confirmation of Roles of Neutrophils in Decreased Sepsis-Induced Mortality by PLD2 Deficiency

Since PLD2 deficiency considerably diminishes mortality induced by sepsis, to identify if PLD2 still exhibits pathogenic effects even in neutrophil-restricted circumstances, an experiment was performed as follows.

9-1. Adoptive Transfer of Neutrophils

For adoptive transfer of neutrophils, at 2 and 24 hours before CLP surgery, 500 μg of αLy6G antibodies (1A8: BioXCell) were injected into each recipient WT mouse. Neutrophil depletion was evaluated using a flow cytometer after neutrophils in peripheral blood were stained with anti-CD11b and anti-Ly-6G, and thus it was confirmed that the recipient neutrophils had been depleted by >99%.

Neutrophil-depleted recipient WT mice were subjected to CLP surgery, and neutrophils (2×10⁶) isolated from WT or PLD2 knockout (KO) bone marrow were intravenously injected into the WT recipient mice. Meanwhile, for in vivo trafficking experiment, neutrophils in WT or PLD2 knockout (KO) BALF were labeled with CFSE (green; 5 μM. Invitrogen), incubated at 37° C. for 15 minutes, and then intravenously injected into the WT recipient mice.

9-2. Comparison of Bacterial Colony Counts

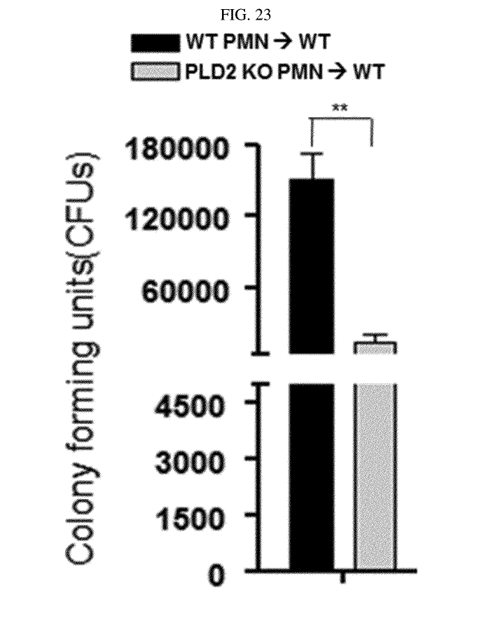

Bacterial colony counts detected from peritoneal fluids of WT recipient mice to which the neutrophils of the WT or PLD2 knockout (KO) mice (donor) were adoptive-transferred according to Example 9-1 were compared by the same method as described in Example 7-1. As a result, as shown in FIG. 23, it can be confirmed that bacterial colony counts in the peritoneal fluids were considerably decreased in recipient mice receiving PLD2 knockout (KO) neutrophils, compared with recipient mice receiving neutrophils of the WT mice.

9-3. Comparison of Apoptosis of Cells

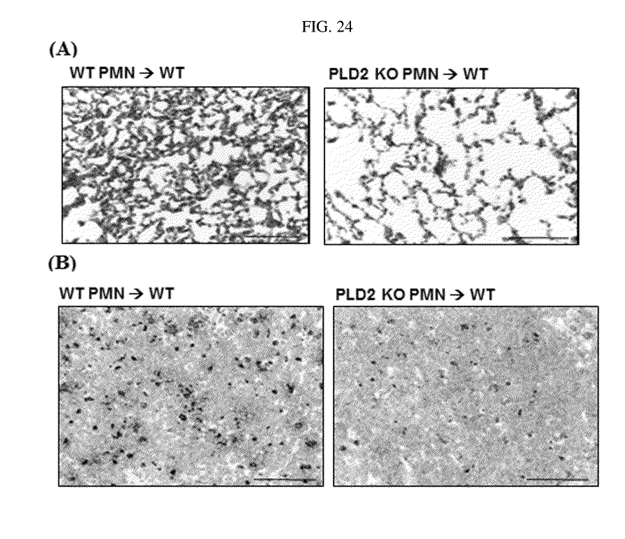

Lung inflammation and spleen cell apoptosis of WT recipient mice to which the neutrophils of the WT or PLD2 knockout (KO) mice (donor) were adoptive-transferred according to Example 9-1 were compared by a TUNEL assay described in Example 6-1. As a result, as shown in FIG. 24, it can be confirmed that the lung inflammation and the spleen cell apoptosis were significantly decreased in CLP-recipient mice receiving neutrophils of the PLD2 knockout (KO) mice, compared with mice receiving neutrophils of the WT mice.

9-4. Comparison of Variations in Inflammatory Cytokine/Chemokine Production

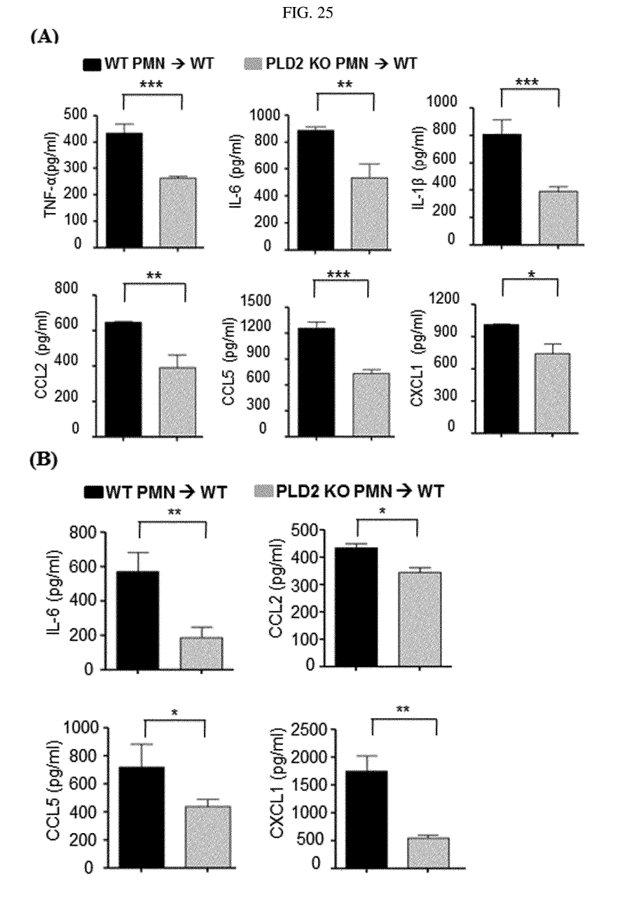

Variations in inflammatory cytokine/chemokine production of WT recipient mice to which the neutrophils of the WT or PLD2 knockout (KO) mice (donor) were adoptive-transferred according to Example 9-1 were compared by the method described in Example 5. As a result, as shown in FIG. 25, levels of proinflammatory cytokines (TNF-α, IL-6, and IL-1β) and inflammatory chemokines (CCL2, CCL5, and CXCL1) in peritoneal fluids and BALFs were significantly decreased in CLP-recipient mice receiving neutrophils of the PLD2 knockout (KO) mice, compared with mice receiving neutrophils of the WT mice.

9-5. Neutrophil Recruitment

In Example 8, total numbers of respiratory tract-infiltrating neutrophils in the PLD2 knockout (KO) CLP mice were considerably higher than those in the WT mice, and therefore it was confirmed that such a phenotype was caused by PLD2 deficiency in neutrophil-restricted circumstances.

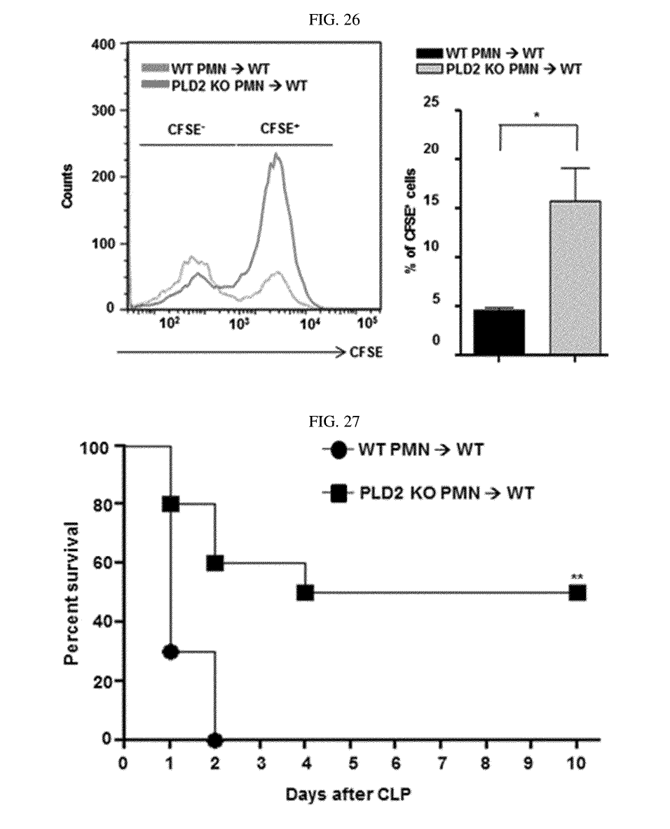

To this end, in vivo trafficking potentials of WT recipient mice to which the CFSE-labeled neutrophils of the WT or PLD2 knockout (KO) mice (donor) were adoptive-transferred according to Example 9-1 were measured by a flow cytometer. As a result, as shown in FIG. 26, it can be confirmed that, compared with mice receiving neutrophils of the WT mice, neutrophils of CLP-recipient mice receiving neutrophils of the PLD2 knockout (KO) mice were more effectively recruited into the respiratory tract.

9-6. Variations of Survival Rates

WT recipient mice to which the CFSE-labeled neutrophils of the WT or PLD2 knockout (KO) mice (donor) were adoptive-transferred according to Example 9-1 were subjected to CLP surgery according to the method described in Example 1-1, and then the variations of survival rates over time were compared. As a result, as shown in FIG. 27, it can be confirmed that, in comparison, CLP-recipient mice receiving neutrophils of the PLD2 knockout (KO) mice had higher survival rates than the mice receiving neutrophils of the WT mice.

The above results showed that the decreased apoptosis caused by sepsis due to PLD2 deficiency was mediated by the neutrophils.

A PLD2 inhibitor according to the present invention exhibits characteristics such as blocking of lung inflammation and liver inflammation, bactericidal activity through induction of NET production, effective maintenance of neutrophil mobility through the blocking of intracellular CXCR2 migration, and blocking of the production of inflammatory cytokines in bacterial infectious disease models, particularly, sepsis models, and thus is expected to be used as a therapeutic agent for sepsis or septic shock.

It would be understood by those of ordinary skill in the art that the above descriptions of the present invention are exemplary, and the exemplary embodiments disclosed herein can be easily modified into other specific forms without changing the technical spirit or essential features of the present invention. Therefore, it should be interpreted that the exemplary embodiments described above are exemplary in all aspects, and are not limitative.

What is claimed is:

1. A method for treating a bacterial infectious disease, comprising:
administering a pharmaceutically effective amount of a pharmaceutical composition comprising a phospholipase D2 (PLD2) inhibitor as an active ingredient to a subject.

2. The method of claim 1, wherein the disease is sepsis or septic shock.

3. The method of claim 1, wherein the PLD2 inhibitor is selected from the group consisting of 5-fluoro-2-indolyl des-chlorohalopemide, N-[2-[1-(3-Fluorophenyl)-4-oxo-1,3,-8-triazaspiro[4.5]dec-8-yl]ethyl]-2-naphthalenecarboxamide, N-{2-[4-oxo-1-phenyl-1,3,8-triazaspiro(4.5)decan-8-yl]ethyl}quinoline-3-carboxamide, N-(2-{4-[2-oxo-2, 3-dihydro-1H-benzo(d)imidazol-1-yl]piperidin-1-yl}ethyl)-2-naphthamide, (1R,2R)—N—([S]-1-{4-[5-bromo-2-oxo-2,3-dihydro-1H-benzo(d)imidazol-1-yl]piperidin-1-yl}propan-2-yl)-2-phenylcyclopropanecarboxamide), and N-[2-[4-(5-chloro-2,3-dihydro-2-oxo-1H-benzimidazol-1-yl)-1-piperidinyl]-ethyl]-4-fluoro-benzamide.

4. The method of claim 1, wherein the composition has bactericidal activity.

5. The method of claim 4, wherein the composition improves peptidylarginine deiminase 4 (PAD4) activity in neutrophils.

6. The method of claim 1, wherein the composition decreased production of inflammatory cytokines.

7. The method of claim 6, wherein the inflammatory cytokine is selected from the group consisting of TNF-α, IL-1β, IFN-γ, IL-17 and IL-23.

8. The method of claim 1, wherein the composition inhibits lymphocyte apoptosis.
